(19) United States
(12) Patent Application Publication
(10) Pub. No.: US 2024/0057926 A1
(43) Pub. Date: Feb. 22, 2024

(54) NEUROFEEDBACK REHABILITATION SYSTEM

(71) Applicant: UNIVERSITY OF SOUTHEN CALIFORNIA, Los Angeles, CA (US)

(72) Inventors: Sook-Lei Liew, Los Anges, CA (US); Octavio Marin-Pardo, Los Angeles, CA (US); Coralie Phanord, Los Angeles, CA (US)

(73) Assignee: UNIVERSITY OF SOUTHEN CALIFORNIA, Los Angeles, CA (US)

(21) Appl. No.: 18/269,656

(22) PCT Filed: Feb. 25, 2022
(86) PCT No.: PCT/US2022/017934

§ 371 Date: Jun. 26, 2023

Related U.S. Application Data
(60) Provisional application No. 63/154,092, filed on Feb. 26, 2021.

(51) Int. Cl. A61B 5/375; A61B 5/00; A61B 5/397; A61B 5/372; A61B 5/256; A61B 5/291; A61B 5/296

(52) U.S. Cl. CPC A61B 5/375; A61B 5/744; A61B 5/397; A61B 5/372; A61B 5/256; A61B 5/291; A61B 5/296; A61B 5/7455; A61B 5/7267; A61B 2560/0431; A61B 2560/0443

(57) ABSTRACT

A plurality of sensors detect neural activity and/or muscle activity of a person. A display renders an image with an avatar that represents the person. A processor moves a portion of the avatar in response to calculations performed on signals corresponding to the neural activity and the muscle activity. The avatar may be caused to move in a manner corresponding to an expected muscle movement of the person. However, due to illness or disease, the person may be unable to fully execute the expected muscle movement. By watching the avatar move in response to efforts to execute an expected muscle movement, the person may train his or her body to more fully execute desired muscle movements, such as through neurofeedback for rehabilitation.

### CROSS-REFERENCE TO RELATED APPLICATIONS

This application is based upon and claims priority to U.S. provisional patent application 63/154,092 entitled “NEUROFEEDBACK REHABILITATION SYSTEM” and filed on Feb. 26, 2021, the entire content of which is incorporated herein by reference.

### STATEMENT AS TO FEDERALLY SPONSORED RESEARCH

This invention was made with government support under grant number W911NNF-14-D-0005 awarded by the U.S. Army Research Office, and K01HD091283 awarded by the National Institutes of Health. The government has certain rights in this invention.

### BACKGROUND

### 1. Field

This disclosure relates generally to neurofeedback systems, and more specifically, to neurofeedback systems for rehabilitation of medical conditions.

### 2. Description of the Related Art

Every year, approximately 800,000 individuals experience a stroke in the US, with related costs exceeding $33 billion. Due to advances in acute stroke care, stroke mortality rates have been decreasing. However, this has resulted in more severe stroke survivors with higher post-stroke rehabilitation and long-term care costs. Research has shown that more rehabilitation therapy leads to better functional outcomes. However, people with moderate to severe motor impairments recovering from stroke (i.e., who cannot move their limbs) are excluded from most therapies and home programs, which require volitional movement. Therapy for this population instead focuses on preparing the individual for permanent dysfunction (e.g., caregiver training, instruction in the use of adaptive equipment). This growing population has the largest negative impacts on quality of life and the highest costs in terms of long-term medical care. There is a need for effective treatments for the approximately 32 million people (and growing) currently living with moderate to severe motor impairments after stroke.

### SUMMARY

In various embodiments, a neurofeedback rehabilitation system is provided. The system may have a plurality of sensors configured to detect neural activity of the brain of a patient and/or electrical activity of a muscle of the patient at different locations, or proxies of these brain and/or muscle signals, derived from other biological signals, including but not limited to hand tracking, grip force, eye tracking, or heart rate. The system may have a display configured to render an image of a portion of an avatar that is representative of a patient or another type of neurofeedback representative of the biological signal measured. The system may have a processor coupled to the plurality of sensors and the display. The processor may be configured to determine a movement of the portion of the avatar based on a coherence between the neural activity and the muscular activity (e.g., corticomuscular coherence), or a proxy for this signal, of the patient at the different locations. The processor may be configured to render, on the display, the movement of the portion of the avatar to provide personalized or customized biofeedback to the patient, which may be adaptative, in response to the patient's performance. The system may also include a virtual reality, augmented reality, or mixed reality headset or other digital display configured to be placed over a head of the patient or in front of the patient and includes the display.

The processor may be configured to do further aspects. For instance, the processor may be configured to (i) determine a ratio of the neural activity at the different locations and/or a ratio of the muscular activity at the different locations and (ii) determine the movement of the portion of the avatar further based on the ratio of the neural activity and the ratio of the muscular activity, based on neuromuscular coherence, or based on a proxy for one of these signals, at the different locations. The processor may be configured to (i) determine a change in the ratio of the neural or muscular activity at the different locations or a change in the ratio of the muscular activity at the different locations and (ii) determine a change in the movement of the portion of the avatar based on the change in the ratio of the neural activity or the change in the ratio of the muscular activity.

The plurality of sensors may include electroencephalography (EEG) sensors that are positioned at various locations on the head, wherein the EEG sensors are configured to detect the neural activity of the brain that corresponds to a movement or likely control of movement of a hand, an arm or a leg of the patient. The system may have a neoprene or other adjustable, customizable sleeve including one or more sensors of the plurality of sensors, wherein the one or more sensors include one or more electromyography (EMG) sensors that are placed or fitted on the neoprene sleeve, wherein the one or more sensors are one or more flexible fabric electrodes or one or more disposable gel electrodes with snaps. The system may have a communication interface configured to couple with a remote computing device that allows a provider to communicate with and monitor the patient, wherein monitoring the patient includes monitoring the detected neural activity of the brain of the patient or the detected muscular activity. The movement of the portion of the avatar may be further based on one or more settings that are configurable by input received from a remote device that is operated by a provider, by user input received via a user interface or is adjusted based on previously-detected neural activity or previously-detected muscular activity.

A portable, modular neurofeedback rehabilitation system is provided. The system may have a remote device configured to allow a provider to monitor or communicate with a patient. The provider may be also represented in the virtual environment as a digital display, as an avatar, or as themselves (e.g., via video). The system may have a plurality of sensors configured to detect neural activity of the brain of a patient, electrical activity of a muscle of a patient, or other biological signal such as muscle force, heart rate, hand tracking, or eye tracking of a patient, at different locations. The system may have a display configured to render an image of a portion of an avatar that is representative of a patient. The system may have a processor coupled to the plurality of sensors and the display. The processor may be configured to (i) determine a movement of the portion of the avatar based on a coherence of the neural activity of the brain and the electrical activity of the muscle of the patient and (ii) render, on the display, the movement of the portion of the avatar to provide biofeedback to the patient that results in an improvement of a condition of the patient or triggers a response by the patient.

In various embodiments of the portable, modular neurofeedback rehabilitation system, the processor is configured to determine the movement of the portion of the avatar further based on one or more settings or thresholds that are personalized to the patient, wherein the remote device is configured to manually or automatically provide or adjust the one or more settings or thresholds that are personalized to the patient based on the patient's performance, possibly using machine learning algorithms to optimize performance and learning, and the processor is configured to receive the one or more settings or thresholds, wherein the one or more settings or thresholds are adjusted over time to facilitate the improvement of the condition of the patient by requiring a stronger response or an increased amount of neural activity or electrical activity by the patient.

The system may also include one or more hand controllers configured to provide vibrotactile or other sensory feedback to the patient in response to the virtual environment surrounding the avatar in the image and the detected neural activity and the detected muscular activity. The plurality of sensors include at least one of an electroencephalography (EEG) sensor that is positioned at a location on a head of the patient, an electromyography sensor (EMG) sensor that is positioned at a location on a body part of the patient, and/or a camera that tracks movement of an arm, a leg, a hand or other body part of the patient, wherein the EEG sensor(s) is configured to detect the neural activity of the brain that corresponds to a movement or likely control of movement of the arm, the leg, the hand or the other body part of the patient and the EMG sensor measures the electrical activity of the muscle of the body part of the patient.

In various embodiments of the system, the processor is configured to determine the movement of the portion of the avatar further based on an amplitude or magnitude of the neural activity or an amplitude or magnitude of the electrical activity generated by the muscle.

A method for providing biofeedback to a patient is provided. The method may include detecting, by a processor and using a plurality of sensors, neural activity of a brain of a patient and/or electrical activity of a muscle of the patient. The method may include determining, by the processor, a movement of a portion of an avatar based on a coherence or relationship between the neural activity of the brain of the patient and/or the electrical activity of the muscle of the patient. The method may include rendering, by the processor and on a display, the movement of the portion of the avatar to provide biofeedback to the patient.

The method may include other aspects as well. For instance, the method may include determining, by the processor, the coherence or the relationship between the neural activity of the brain of the patient and the electrical activity of the muscle of the patient. The method may include determining, by the processor and using a patient training model, one or more settings or thresholds that represent an expected level of coherence between the neural activity and the muscular activity or an expected level of neural activity and muscular activity to generate the movement of the portion of the avatar. The method may include determining, by the processor, the movement of the portion of the avatar further based on the one or more settings or thresholds. The method may include (i) obtaining, by the processor, patient data representative of a population of patients that require physical or occupational therapy after a condition including a stroke, wherein the patient data includes a level of coherence of neural activity of brains of the patients and electrical activity of muscles of the patients and corresponding settings or thresholds for movement of the portion of the avatar that induced improvement in movement of the patient and (ii) generating, by the processor, the patient training model using a machine learning algorithm based on the obtained patient data.

The method may include determining, by a processor, a ratio of the neural activity at the different locations and a ratio of the muscular activity at the different locations, wherein determining the movement of the portion of the avatar is further based on the ratio of the neural activity and the ratio of the muscular activity at the different locations.

The method may include communicating, by the processor, with a remote computing device to monitor the patient via the remote computing device, wherein monitoring the patient includes monitoring the detected neural activity of the brain of the patient or the detected muscular activity.

### BRIEF DESCRIPTION OF THE DRAWINGS

Other systems, methods, features, and advantages of the present invention will be or will become apparent to one of ordinary skill in the art upon examination of the following figures and detailed description. Additional figures are provided in the accompanying Appendix and described therein.

FIG. 1 shows an example embodiment of hardware aspects of a neurofeedback rehabilitation system, in accordance with various embodiments;

FIG. 2 shows an example logical architecture of aspects of the neurofeedback rehabilitation system, in accordance with various embodiments;

FIG. 3 shows an example sensor of the neurofeedback rehabilitation system, in accordance with various embodiments;

FIG. 4 shows an example method of neurofeedback rehabilitation, in accordance with various embodiments; and

FIG. 5 shows an example method of neurofeedback rehabilitation with machine learning, in accordance with various embodiments.

### DETAILED DESCRIPTION

This disclosure presents a system, apparatus, method for a low-cost, modular neurofeedback rehabilitation (or “neurofeedback rehabilitation system”). The neurofeedback rehabilitation system provides a brain/muscle-computer interface for neurorehabilitation. The neurofeedback rehabilitation system may be augmented with digital gaming technologies, such as virtual or augmented reality, and combined with a brain/muscle-computer interface to motivate engaging and frequent exercise to promote neural recovery during physical, occupational, or other rehabilitation therapies. The neurofeedback rehabilitation system may be employed on patients with wide-ranging conditions, ailments, diseases and/or impairments including in chronic stroke patients, orthopedic patients, patients with Alzheimer's disease, Parkinson's disease, Cerebral Palsy, individuals post-surgery or any other patient with a neurological, cognitive, or physiological disorder that requires rehabilitation to improve sensorimotor skills and/or motor, sensory or cognitive assessment or detection.

The neurofeedback rehabilitation system provides brain and/or muscle biofeedback in the form of engaging and immersive games using head-mounted virtual reality devices, augmented reality devices, or a display screen of a computer, a tablet, or a mobile device. The system may be used in research, clinical, or at-home settings with telerehabilitation (e.g., video telehealth conference with a therapist). The system may be customizable and contain different user-friendly graphical user interfaces for researchers, clinicians and patients and include one or more applications, such as games that can be selected and parameterized based on the patient's needs. The system may be used for a variety of rehabilitation and training purposes, including in healthy individuals or individuals with brain injury or neuromuscular disorders, such as cerebral palsy, Parkinson's, mild cognitive impairment, Alzheimer's disease, etc., to modulate brain and/or muscle activity in a safe, engaging, and non-invasive and non-pharmacological manner. The system provides adaptive, customizable biofeedback in these activities and is further described herein.

Importantly, the neurofeedback rehabilitation system provides a closed-loop brain computer interface that provides customizable, targeted neurofeedback to re-train and recover damaged neural networks, in the absence of volitional movement, resulting in improved sensorimotor control. An individual's own neuromuscular commands are sensed and used with corresponding augmented, but believable, embodied feedback of one's “own” movements in VR, engaging the brain's action observation network and essentially tricking the brain into thinking the affected limb is moving again. Research associated with development of the system, apparatus, and method has shown that seeing one's limb represented in VR leads to activity in the damaged motor cortex and evoke changes in overt behaviors. The system, apparatus, and method improves sensorimotor function, strengthens damaged corticomuscular connections, and leads to improved quality of life for patients with stroke. The neurofeedback rehabilitation system can be flexibly adapted to each patient's specific needs. The neurofeedback rehabilitation system can provide neurofeedback of brain, muscle or hand movement signals and can be customized and calibrated to each person's individual neuromuscular profile.

The system can provide feedback in virtual reality, augmented reality, or via a computer screen. The neurofeedback rehabilitation system is easy to use across hospital, clinic and/or home settings. Notably, the neurofeedback rehabilitation system can be used at home by severe stroke patients by themselves, without the help of a caregiver, with customized adaptations such as a neoprene sleeve to apply the EMG electrodes using only one hand. Finally, though one reason for development of the neurofeedback rehabilitation system is for stroke rehabilitation, the modular technology is also easily extended to provide tailored neurofeedback for populations with other neurological and neurodegenerative disorders, such as people with cerebral palsy, Parkinson's disease, or traumatic brain injury, or cognitive rehabilitation for people with cognitive impairment or Alzheimer's disease, as mentioned above.

One effective method for improving upper limb function, which could be combined with telerehabilitation, is the reinforcement of muscle activity using electromyography (EMG) biofeedback. Muscle biofeedback has been shown to reduce spasticity and improve post-stroke arm function, motor control, muscle activity, and strength. Biofeedback training to avoid unintended simultaneous activation of antagonist muscle groups may be particularly beneficial for reducing unnecessary co-contractions that impede functional motor control.

Portable systems for at-home use improve accessibility and training time with EMG biofeedback. However, proper implementation of home-based EMG biofeedback is critical to prevent low participant adherence, avoid high costs, and account for limitations in terms of required physical space, time, and technical literacy. Also, the ability to track patient progress in real-time and the continued involvement of a clinician in the intervention are key factors that could improve patient motivation and adherence to at-home rehabilitation programs.

The system herein provides biofeedback of brain and/or muscle activity on an electronic screen, in immersive virtual reality (VR) with a head-mounted display (HMD). The system herein also incorporates a telerehabilitation component for live video and audio conferencing with a clinician who meets regularly with the participant to monitor progress. The clinician can also provide technical support, ensure the electrodes are placed correctly, and monitor biological signals (e.g., EMG signals) in real-time to ensure adequate signal quality. In addition, the system can use a portable laptop with low-cost EMG sensors for greater affordability and accessibility in the home environment. Lastly, the system includes gamified elements to encourage greater engagement, motivation, and adherence to a home-based program.

This disclosure will present a detailed description of the system and also an example case study. In one example case study, the system is utilized by a 67-year-old male stroke survivor, 11 years after stroke onset for a utilization period of 10 weeks. The participant had upper extremity hemiparesis, was not taking anti-spasticity medication, had no receptive aphasia, had corrected vision, and did not have a secondary neurological disease. The participant had less than 15 degrees of active wrist or finger extension in the more impaired hand and was unable to grasp and release a ball unassisted.

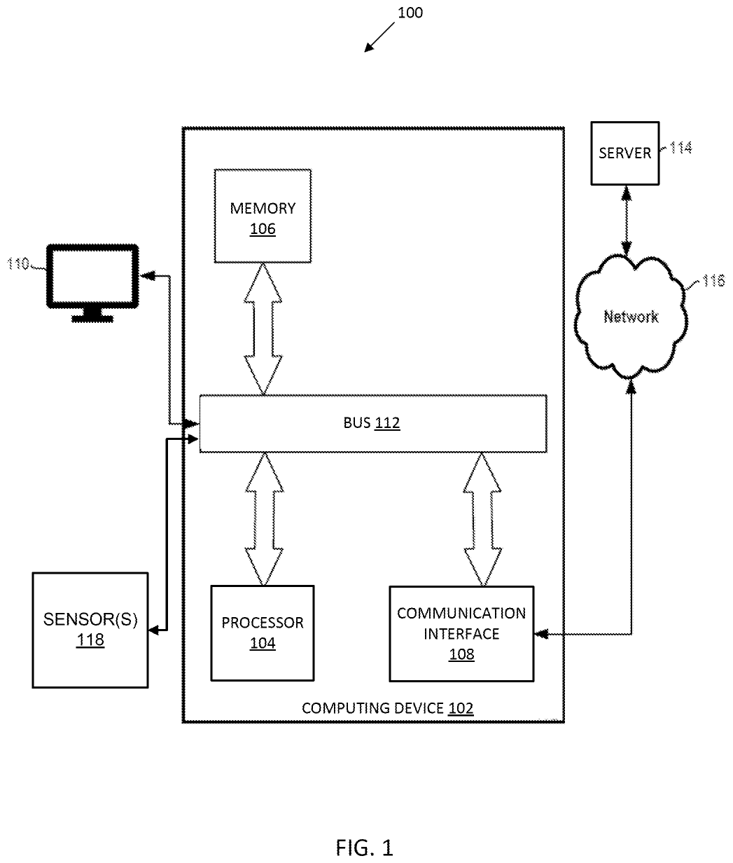

Directing attention now to FIG. 1, an example embodiment of hardware aspects of a neurofeedback rehabilitation system 100 is shown. The system may include a computing device 102, such as a laptop computer, a table computer, a smartphone, or another computing system. The computing device 102 may include one or more processors 104, a memory 106 and/or a bus 112 and/or other mechanisms for communicating between the one or more processors 104. The one or more processors 104 may be implemented as a single processor or as multiple processors. The one or more processors 104 may execute instructions stored in the memory 106 to implement the applications, such as the methods provided herein.

The one or more processors 104 may be coupled to the memory 106. The memory 106 may include one or more of a Random Access Memory (RAM) or other volatile or non-volatile memory. The memory 106 may be a non-transitory memory or a data storage device, such as a hard disk drive, a solid-state disk drive, a hybrid disk drive, or other appropriate data storage, and may further store machine-readable instructions, which may be loaded and executed by the one or more processors 104.

The memory 106 may include one or more of random access memory (“RAM”), static memory, cache, flash memory and any other suitable type of storage device or computer readable storage medium, which is used for storing instructions to be executed by the one or more processors 104. The storage device or the computer readable storage medium may be a read only memory (“ROM”), flash memory, and/or memory card, that may be coupled to a bus 112 or other communication mechanism. The storage device may be a mass storage device, such as a magnetic disk, optical disk, and/or flash disk that may be directly or indirectly, temporarily or semi-permanently coupled to the bus 112 or other communication mechanism and used be electrically coupled to some or all of the other components within the computing system 100 including the memory 106, the user interface 110 and/or the communication interface 108 via the bus 112.

The term “computer-readable medium” is used to define any medium that can store and provide instructions and other data to a processor, particularly where the instructions are to be executed by a processor and/or other peripheral of the processing system. Such medium can include non-volatile storage, volatile storage and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

The system 100 may include a user interface 110. The user interface 110 may include an input/output device. The input/output device may receive user input, such as a user interface element, a button, a dial, a microphone, a keyboard, or a touch screen, or a device that reads in biological signals from the user, and/or provides output, such as a display, a speaker, an audio and/or visual indicator, tactile feedback, a VR headset, an augmented reality device, or a refreshable braille display. The display may be a computer display, a tablet display, a mobile phone display, an augmented reality display or a virtual reality headset. The display may output or provide a virtual environment that mimics actions of the patient and/or provide information regarding the neural or muscular activity of the patient or other information.

The user interface 110 may include an input/output device that receives user input, such as a user interface element, a button, a dial, a microphone, a keyboard, or a touch screen, and/or provides output, such as a display, a speaker, headphones, an audio and/or visual indicator, tactile feedback, a VR headset, an augmented reality device, or a refreshable braille display. The speaker may be used to output audio associated with the audio conference and/or the video conference. The user interface 118 may receive user input that may include configuration settings for one or more user preferences, such as a selection of joining an audio conference or a video conference when both options are available and/or for joining or launching the game, the telehealth session, or the like. In some instances, the user interface 110 includes one or more hand controllers to provide vibrotactile feedback to the patient in response to the virtual environment surrounding an avatar in an image on the user interface 110 and detected neural activity and/or detected muscular activity detected by sensors 118 (discussed below).

The system 100 may have a network 116 that connects to a server 114. The network 116 may be a local area network (LAN), a wide area network (WAN), a cellular network, the Internet, or combination thereof, that connects, couples and/or otherwise communicates between the various components of the system 100 with the server 114. The server 114 may be a remote computing device or system that includes a memory, a processor and/or a network access device coupled together via a bus. The server 114 may be a computer in a network that is used to provide services, such as accessing files or sharing peripherals, to other computers in the network.

The system 100 may include a communication interface 108, such as a network access device. The communication interface 108 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). The communication interface may transmit data to and receive data among the different components.

The server 114 may include a database. A database is any collection of pieces of information that is organized for search and retrieval, such as by a computer, and the database may be organized in tables, schemas, queries, reports, or any other data structures. A database may use any number of database management systems. The information may include real-time information, periodically updated information, or user-inputted information.

The system 100 may include sensors 118. The sensors may include one or more hand-held controllers, electromyography (EMG) electrodes, and/or electroencephalography (EEG) electrodes. A combination of multiple types of sensors may be included. The sensors 118 may include a processor board, such as a Teensy 4.0 development board (PJRC.COM LLC, Sherwood, OR, USA). The sensors 118 may include a pair of Myoware muscle sensors (Advancer Technologies LLC, Raleigh, NC, USA) to acquire signals from wrist extensor and flexor muscle groups. In various instances, sensors 118 include 3D-printed cases to enclose hardware components to provide electrical insulation and improve durability.

The sensors may be implemented so that each muscle sensor may amplify a signal registered between a pair of differential electrodes. The processor board may digitize the signals send them to other aspects of the system 100, such as the processor 104 through a USB cable via serial communication. A software application such as a C#application may be connected to the board by the processor. Signals may be streamed to a local network 116. In one example, this configuration facilitates acquisition of up to 4 or more channels of EMG signals at 2000 Hz with 12-bit ADC resolution. In various embodiments, up to 8 or more channels of EEG are acquired. Moreover, sensors may include cameras, motion sensors, or other motion tracking devices. For instance, up to 2 or more hand tracking sensors may be implemented. In various embodiments, sensors may include sensors to measure force during grip, wrist extension, or other movements, or other biological signals.

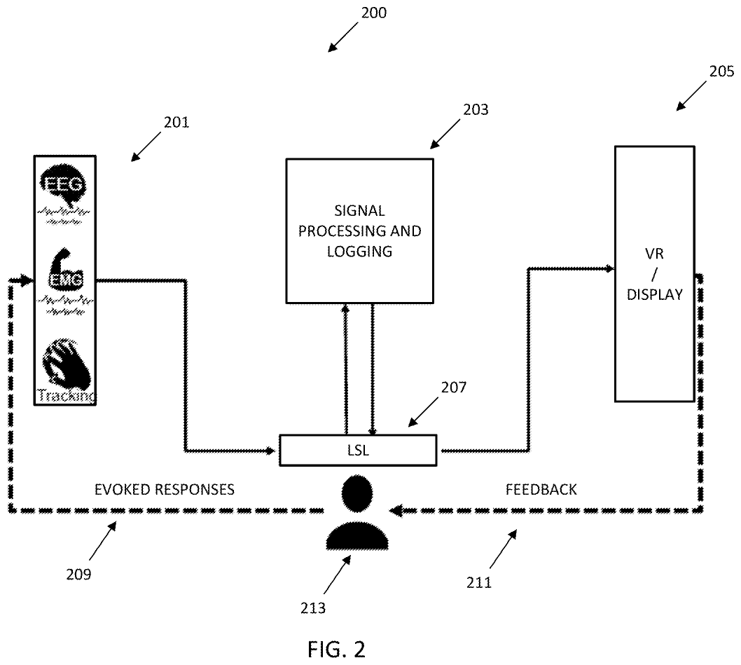

Turning to FIG. 2, the system 100 may have various logical architectures, one example of which is represented here. The system 100 may have a logical architecture 200 with an interfacing aspect 201. The interfacing aspect may comprise data structures corresponding to inputs from sensors 118 (FIG. 1). For instance, motion tracking of movements perceived by a motion tracking device, EMG data collected from EMG electrodes, or EEG data collected from EEG electrodes may be captured. EEG sensors may be positioned at various locations on the head to detect the neural activity of the brain that corresponds to a movement or likely control of movement of a hand, an arm, a leg, or other body part of the patient. EMG sensors may be positioned adjacent a muscle. Motion tracking sensors may be positioned to have a view of a movement or likely movement of a hand, arm, leg, or other body part of the patient.

The logical architecture 200 may include an interaction aspect 205. An interaction aspect 205 may include a human-perceptible output on the user interface 118 (FIG. 1). For instance, a visual display screen may show a game, or a VR session with an avatar, or the like. The architecture may include a software system for unified collection data, such as using lab streaming layer (LSL) 207. LSL may provide a software application protocol that may transmit data between and among modules for processing, game interaction, and storage. By connecting modules through LSL, the architecture facilitates the functional independence of each component. This is advantageous for continued development since different configurations of sensors, processing pipelines, and environments can be implemented without necessitating updates to other modules. The modular system allows for the input of electroencephalography, EMG or movement data, and visual output to a screen or HMD-VR system as well as proprioceptive feedback via handheld controllers. Additionally, a script facilitates real-time visualization of the digitized EMG signals. The system may also include a processing aspect 203. A processing aspect 203 receives data from the interfacing aspect 201 and generates outputs for the interaction aspect 205 based on the data from the interfacing aspect 201. A human user 213 is also illustrated. In an example use case, the interaction aspect 205 provides visual and/or vibrotactile feedback 211 to a user 213 corresponding to an evoked response 209 of a user's nervous or musculoskeletal system, which are detected by the interfacing aspect 201.

The logical architecture may include custom scripts. For instance, a Matlab script running on the processing aspect 203 may process EMG signals in real-time and use them for game control of a game shown on an interaction aspect 205. Digitized signals may be filtered, rectified, and normalized to a prerecorded maximum grip. A value of normalized activity close to 0 corresponds to no volitional activity registered by the sensor and a value close to 1 represents an amplitude similar to that seen during an attempted grip. The clinician or researcher can modify the specifics of the processing algorithms based on the patient needs.

The logical architecture may facilitate the playing of games by attempted volitional activity. Games may be developed in the Unity game engine to provide feedback in the form of different games and stream game interactions (e.g., current score and trial number) through the LSL protocol. The games will be discussed further in a section below.

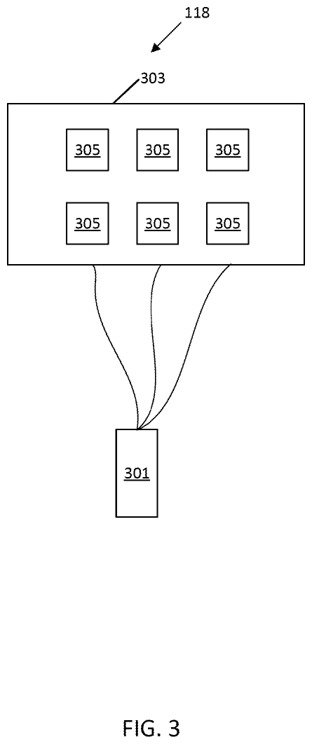

Referring now to FIG. 3, an example sensor 118 (FIG. 1) is illustrated. An example sensor 118 may include electrodes 305. Electrodes 305 may be placed on a body adjacent a muscle and detect electrical signals associated with muscle activation by a wearer. The electrodes 305 may be attached to a customizable wearable sleeve or bands 303 configured to hold the electrodes 305 in position. The electrodes 305 may provide detected electrical signals to an acquisition board 301. An acquisition board 301 may combine signals from multiple electrodes 305 and amplify, filter, or otherwise process the signals. The acquisition board 301 may digitize the signals and provide the signals to the computing device 102 (FIG. 1). The wearable sleeve 303 may be a neoprene or other adjustable, customizable sleeve. The electrodes 305 may be flexible fabric electrodes or one or more disposable gel electrodes with snaps.

Turning now with combined reference to FIGS. 1, 2, and 3, a nonlimiting example implementation of a neurofeedback rehabilitation system 100 is provided. A neurofeedback rehabilitation system 100 may have a plurality of sensors 118 configured to detect neural activity of a brain of a patient and/or electrical activity of a muscle of the patient at different locations. The system 100 also has a display 110, such as a computer monitor, a smartphone screen, or a VR headset, configured to render an image of a portion of an avatar that is representative of a patient. The system may also include a processor 104 coupled to the plurality of sensors 118 and the display 110. The processor 104 may be configured to determine a movement of the portion of the avatar based on a coherence between the neural activity and the muscular activity of the patient at the different locations. As used herein, a coherence means a degree to which a muscle activity that is exhibited by the patient is consistent with a neural activity of a patient endeavoring to cause the muscle activity. Such correspondence is often impeded by medical conditions such as a stroke, and by engaging in training sessions with the neurofeedback rehabilitation system, function may become restored so that the coherence improves over time. The processor 104 may also be configured to render, on the display 110, the movement of the portion of the avatar to provide personalized or customized biofeedback to the patient. The display 110 may be various types of headsets. For instance, the display 110 a virtual reality, augmented reality, or mixed reality headset or other digital display configured to be placed over a head of the patient or in front of the patient and includes the display.

When determining the movement of the avatar, the processor 104 may perform various calculations. A ratio calculation was previously mentioned. One ratio calculation may be to determine a ratio of the neural activity at the different locations and/or a ratio of the muscular activity at the different locations and then determine the movement of the portion of the avatar further based on the ratio of the neural activity and the ratio of the muscular activity, as well as the neuromuscular coherence, at the different locations. A further ratio calculation may be performed. A change in the ratio of the neural activity at the different locations or a change in the ratio of the muscular activity at the different locations may be calculated and the processor may determine a change in the movement of the portion of the avatar based on the change in the ratio of the neural activity or the change in the ratio of the muscular activity. The rendered movement of the portion of the avatar provides biofeedback to the patient that results in an improvement of a condition of the patient or triggers a response by the patient. The processor 104 may be configured to determine the movement of the portion of the avatar further based on an amplitude or magnitude of the neural activity or an amplitude or magnitude of the electrical activity generated by the muscle.

In various instances, machine learning may also be incorporated. For instance, the processor 104 may obtain patient data representative of a population of patients that require physical or occupational therapy after a condition including a cardiovascular stroke. This patient data may include a level of coherence of neural activity of brains of the patients and electrical activity of muscles of the patients and corresponding settings or thresholds for movement of the portion of the avatar that induced improvement in movement of the patient. The processor may generate the patient training model using a machine learning algorithm based on the obtained patient data.

The processor 104 may also be coupled with a memory 106. The memory may be configured to store the detected neural activity of the brain of the patient or the detected electrical activity of the muscle of the patient at the different locations. The display 110 may display this detected neural activity or the detected muscular activity over a period of time. The memory 106 may store a plurality of software applications that each provide different virtual environments for the avatar to operate within and the plurality of sensors 118 may be integrated with the plurality of software applications. The detected neural activity or the detected muscular activity may correspond to movement of the portion of the avatar in the different virtual environments. In some instances, the integration of the plurality of sensors 118 with the plurality of software applications and the display is platform and hardware independent or platform and hardware agnostic.

The processor 104 may be coupled with a communication interface 108. The communication interface 108 may couple with a remote computing device such as a server 114 over a network 116 to allow a provider to communicate with and monitor the patient. Monitoring the patient may include monitoring the detected neural activity of the brain of the patient or the detected muscular activity. Thus, being in contact with a remote device, the processor 104 may render an avatar consistent with additional instructions or settings from the remote device. For instance, the movement of the portion of the avatar is further based on one or more settings that are configurable by input received from a remote device that is operated by a provider, by user input received via a user interface or is adjusted based on previously-detected neural activity or previously-detected muscular activity.

Having introduced a system, a logical architecture of aspects of the system, and an example sensor for the system, now is a convenient time to discuss example games that may be played as a part of neurological rehabilitation using a neurofeedback rehabilitation system. One example game may be called “SeeEMG.” This game provides quick visual feedback of real-time extensor and flexor muscle activity as independent continuous streams. After applying the signal processing steps described herein, EMG signals are plotted in each frame as a continuous line for each of the muscles. The participant is allowed to select one of two configurations. Option 1 displays EMG smoothed with a 250-ms Hann window, down-sampled to the refresh-rate of the screen. This option uses a heatmap color scale to map high amplitudes in red and low amplitudes in blue. The default range sets “high” as the same amplitude as the maximum grip used to calibrate the system, and “low” as the lowest amount of activity (typically, none) recorded during calibration. This range can be dynamically adjusted by the clinician based on the average amplitude of the signals to ensure the whole range of colors is rendered. Option 2 displays rectified EMG, down-sampled to the refresh rate of the screen, as white lines. A dynamic green horizontal line is also plotted to represent the maximum amplitude reached during that session.

Another example game may be called “SkeeBall.” This game improves wrist extensor activity and control while reducing abnormal coactivation of wrist flexors during active extension attempts. In this game, the ratio of activity from the wrist extensors and flexors is used to move a ball to different targets. First, the game applies the signal processing steps described herein. Then, the game calculates an extensor ratio (ER) value (ER=extensor/(extensor+flexor)), defined as the sum of the mean extensor activity divided by the sum of the mean activity from extensor and flexor muscles. ER values closer to 1 indicate more individuated extension, closer to 0.5 more coactivation, and closer to 0 more individuated flexion. Other effective methods to calculate the contribution of antagonist activity in different movements and muscle groups may be implemented.

In an example embodiment, the extensor ratio calculation is chosen for its simplicity, low computational requirements, and its ability to capture the relationship of two antagonistic muscles. This ratio is advantageous to encourage wrist extension without simultaneous unintended wrist flexion. For each trial, a score is assigned as a function of the ER value and a probability likelihood. In one example set of scores and probability likelihoods, an ER value of 0.7 would result in 20 points in 60% of trials and 30 points in 40% of trials. Calculated point values trigger the corresponding animation of the hand hitting the ball and the ball moving to the appropriate ring. At the end of the game, the final score is calculated as the cumulative points for each trial. Importantly, the probability values are only used to determine the score of the trial. That is, a calculated value of individuated extension (ER >0.5) will always be positively reinforced, providing higher scores to higher individuation values.

A still further example game may be “Blinko.” This game is configured to improve both wrist extensor and wrist flexor activity while reducing abnormal coactivation. Similar to SkeeBall, the game uses the ratio of activity from the wrist extensors and flexors, the extensor ratio (ER), to move a round flat disc, or chip, across the top of a board. The board is modeled after the popular gameshow chance game Plinko. The board consists of rows of pegs, with each row offset from the one above it. At the bottom of the board, there are nine slots that represent points. These are labeled as follows: $100, $500, $1000,$0, $10,000, $0, $1000, $500, and $100. When a chip is dropped at a slot between two pegs at the top of the board, the chip is diverted by the slots below it. Thus, the chip may take any number of paths to the bottom and land in any of the nine slots. For each trial, the player attempts to move the chip right with wrist extension or left with wrist flexion. Then, the player drops the chip and the points assigned correspond with the bottom slot where the chip landed. At the end of the game, the final score is calculated as the cumulative points for each trial. Score likelihoods are programmed for the SkeeBall game as a function of the calculated extensor ratio (ER).

In connection with playing a game in a telerehabilitation session, the system 100 (FIG. 1) may include a video or audio interaction with a trained clinician. The trained clinician may administer a series of behavioral assessments to quantify the participant's baseline level of impairment to determine training parameters, e.g., training duration, rest intervals, and the number of repetitions per game. These parameters are set by the clinician or researcher and used for remote training sessions but can be modified on a regular basis as sessions advance. Then, the participant undergoes a detailed orientation showing them step-by-step how to use the system and how to properly place the sensors. Alternatively, if it is unfeasible to do all procedures in person (e.g., due to logistics or social distancing protocols), the orientation can also be performed using the incorporated video conferencing application. Training sessions can be done by the participant alone or remotely monitored by the clinician or researcher. In a guided session, the screen can be configured, for example, to view real-time performance of a SkeeBall game, the participant's electrode placement and body movements, the clinician, and real-time EMG signals all at once. Video meetings with the occupational therapist can take place as often as needed. The participant and clinician also communicate via email. Regular contact with the clinician helps maintain participant engagement and adherence.

In one example embodiment, the system includes a laptop computer with all necessary programs preloaded, configured, and displayed in an easy-to-use manner, a pair of EMG sensors with an enclosed acquisition board, and a package of disposable electrodes. When participants first receive the equipment, they are also given a printed manual of how to use the system. For enhanced simplicity and consistency, each session starts with instructional videos that remind the participant how to start a telerehabilitation session, plug in the acquisition device, and position the sensors over the targeted muscles. Then, a calibration video guides the participant through wrist and hand movements, including gross grasp, wrist extension, and wrist flexion.

Upon completion of the recording, a Matlab script filters and rectifies the signals, as described herein, and calculates the mean amplitudes during the grip. These values are later used as a calibration to normalize real-time signals. Then, the participant selects which game they will play and sets visual configurations for the game, e.g., color-coded signals or arm model. Once the participant is ready, the game starts. The number of repetitions per game, rest periods between trials, and the duration of gameplay is determined by the researcher or clinician in collaboration with the participant. These parameters are influenced by fatigue, spasticity, endurance, and other personal factors. Finally, after completion of each training session, de-identified recorded data is securely synchronized to the clinician's or researcher's computer.

In one example use case, the system was used by a person who had experienced a stroke. He completed 40 sessions that spanned 10 weeks of training. For each session, the participant completed 5 blocks of 20 repetitions using the SkeeBall game, described above, for 100 trials per session, which took approximately 1 h per session. All familiarization and verification of sensor placement was performed remotely via videoconferencing with the clinician, who used the real-time EMG signal tracking to assess electrode placement.

For real-time online analysis, digitized EMG signals were bandpass filtered between 150 and 450 Hz, rectified, and normalized to the calculated activity during the grip attempt. Then, ER values were calculated to provide positive reinforcement of individuated extension using the SkeeBall game, as described above. Importantly, the relatively higher frequency range was chosen to account for possible artifacts induced by motion, crosstalk, and increased susceptibility to environmental noise. In further embodiments, high pass frequency filters may be implemented at the 100 Hz range or above to account for such artifacts and obtain better estimations of force, joint stability, motor unit recruitment, intermuscular coherence, and corticomuscular coherence.

For offline analysis of the recorded sessions, trial data from the game and EMG signals were processed with a custom script in Matlab. First, trial number and EMG signals were interpolated and down-sampled, respectively, to 1000 Hz. Then, signals were processed with the same pipeline as the online data; that is, bandpass filtered between 150 and 450 Hz, full-wave rectified, and normalized to the session's recorded calibration with values of maximum activity within a 250-ms moving window. Then, values of averaged activity were calculated from the first 2 s of each movement attempt trial. To account for the within-session variability and evaluate how these activity patterns changed over time, we averaged the activity of the 100 trials of each session. Finally, Pearson's correlations may be used to test for significant changes in EMG signals and game performance across 28 training sessions. Some sessions may be excluded from the analysis due to excessive noise in the recording or missing data. Session averages can be examined for normalized extension activity, flexion activity, and ER with custom scripts in R.

The participant with stroke used the system for 40 sessions over 10 weeks, with 100% adherence to the requested protocol. In terms of user experience, the participant reported no perceived discomfort, pain, or fatigue, and there were no adverse events during any sessions using the system. The average session duration was 40 min, including about 10 min for setup, 30 min of repeated practice (5 blocks of 20 trials per session), and 1-2 min of rest between blocks of trials. Qualitatively, after 40 sessions, values of calculated individuated activity of the extensor muscle showed a significant increase over time (rho=0.59, p<0.001). Normalized activity for the extensor muscle appeared to increase over time, while flexor activity decreased. However, these individual changes were not statistically significant for either the extensor (rho=0.27, p=0.164) or the flexor (rho=−0.34, p=0.071) muscles. Similarly, while changes in game performance improved, this change did not reach statistical significance (rho=0.29, p=0.06). Finally, the participant reported positive changes in motor function, e.g., increased extension at the interphalangeal joints and decreased frequency of muscle spasms, as well as improved overall quality of life, e.g., improved quality and duration of sleep. The participant also reported enjoyment of using the system and requested to keep it after the testing period concluded. Further participants also demonstrated increased corticomuscular coherence and improved wrist extension, in addition to the aforementioned changes and enjoyment of using the system.

This disclosure has demonstrated that ratios of muscle activity can be acquired in an individual with stroke using low-cost sensors. Using low-cost sensors can result in similar results to research-grade sensors for low forces during an isometric task.

The system was feasible, safe, and enjoyable for 40 1-h sessions over 10 weeks of training. Notably, the participant used the system at home without any in-person instruction or guidance, relying only on telerehabilitation video calls and emails with the clinician and research team. The participant reported high satisfaction with the system. The participant also demonstrated significantly increased extensor activity while decreasing flexor activity over time. The system may be further improved with enhanced calibration algorithms that account for placement variability across sessions as well as processing pipelines to help personalize the biofeedback for each individual. For example, including a higher number of sensors would allow the use of algorithms to detect the best combination of sensors.

The discussion includes a laptop computer with specifications that allowed adequate real-time processing of the EMG signals and simultaneous rendering of the feedback game, along with video conferencing. However, various embodiments may use lower-cost hardware, such as a tablet or cellular phone, instead of a full laptop. Various other calibrations may be implemented, for instance, normalizing the recorded activity with the maximum amplitude registered during any attempted movements, as opposed to specifically using an attempted grip. Such calibrations would allow system usage to extend to participants without the ability to grasp an object.

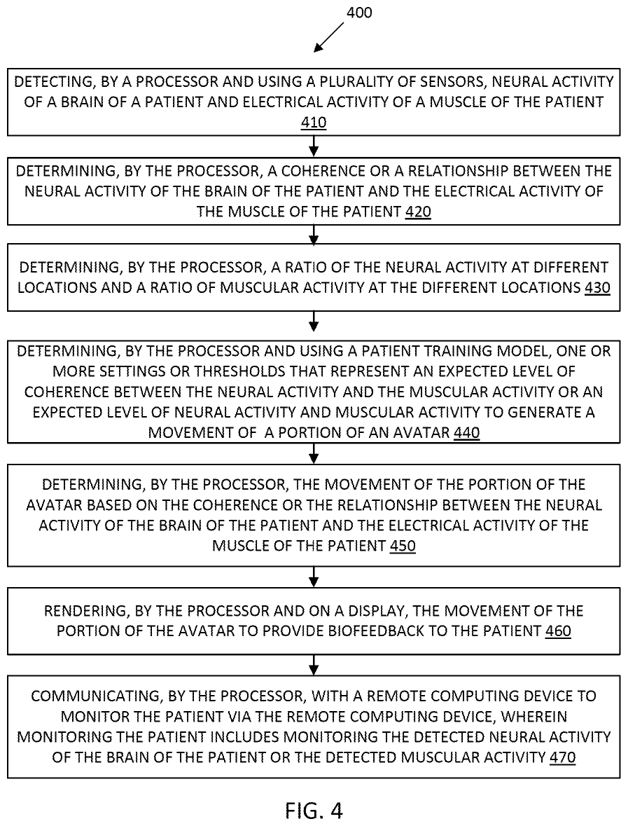

Finally, turning to FIG. 4, a method is provided. The system, logical architecture, and sensors discussed above may be utilized in connection with a method for providing biofeedback to a patient 400. The method may include detecting, by a processor and using a plurality of sensors, neural activity of a brain of a patient and/or electrical activity of a muscle of the patient (block 410). The method may include determining, by the processor, a coherence or a relationship between the neural activity of the brain of the patient and the electrical activity of the muscle of the patient (block 420). The method may include determining, by the processor, a ratio of the neural activity at different locations and a ratio of muscular activity at the different locations (block 430). The method may include determining, by the processor and using a patient training model, one or more settings or thresholds that represent an expected level of coherence between the neural activity and the muscular activity or an expected level of neural activity and muscular activity to generate a movement of a portion of an avatar (block 440). The method may include determining, by the processor, the movement of the portion of an avatar based on the coherence or the relationship between the neural activity of the brain of the patient and the electrical activity of the muscle of the patient (block 450). The movement of the portion of the avatar may be further based on the one or more settings or thresholds. The movement of the portion of the avatar may be further based on the ratio of the neural activity and the ratio of the muscular activity at the different locations. The method may include rendering, by the processor and on a display, the movement of the portion of the avatar to provide biofeedback to the patient (block 460). The method may include communicating, by the processor, with a remote computing device to monitor the patient via the remote computing device, wherein monitoring the patient includes monitoring the detected neural activity of the brain of the patient or the detected muscular activity (block 470). In various embodiments, monitoring the detected neural activity or the detected muscular activity may include adaptive feedback such that performance on each trial (e.g., success, failure, distance from target, etc.) can be implemented to adjust a difficulty setting for subsequent trials (e.g., subsequent efforts to move the portion of the avatar).

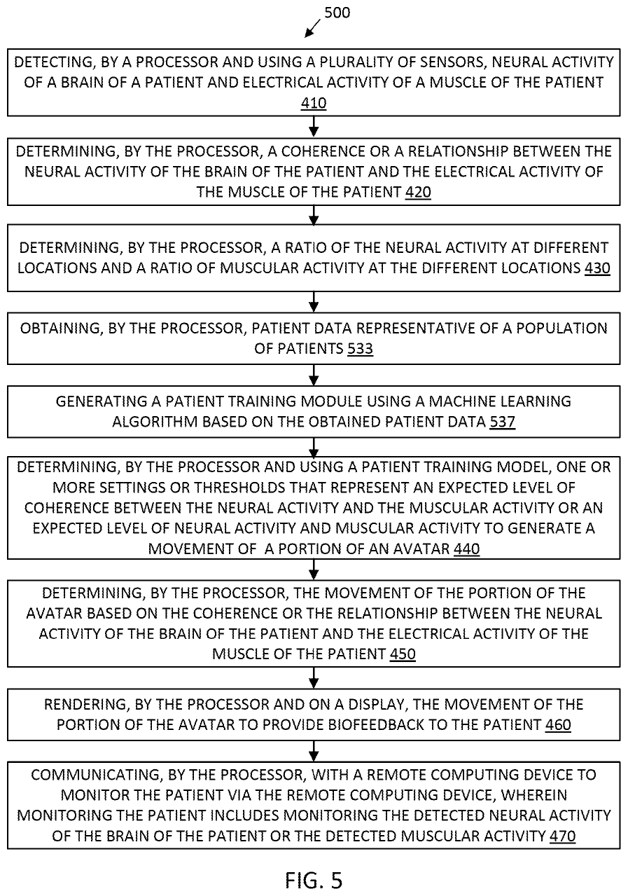

Turning to FIG. 5, a further method with machine learning enhancements is provided. The system, logical architecture, and sensors discussed above may be utilized in connection with a method for providing biofeedback with machine learning to a patient 500. The method may include detecting, by a processor and using a plurality of sensors, neural activity of a brain of a patient and/or electrical activity of a muscle of the patient (block 410). The method may include determining, by the processor, a coherence or a relationship between the neural activity of the brain of the patient and the electrical activity of the muscle of the patient (block 420). The method may include determining, by the processor, a ratio of the neural activity at different locations and a ratio of muscular activity at the different locations (block 430). The method may include, obtaining, by the processor, patient data representative of a population of patients (block 533). The patients may be patients that require physical or occupational therapy after a condition including a stroke. The patient data may include a level of coherence of neural activity of brains of the patients and electrical activity of muscles of the patients and corresponding settings or thresholds for movement of the portion of the avatar that induced improvement in movement of the patient. The method may also include generating a patient training model using a machine learning algorithm based on the obtained patient data (block 537). The method may include determining, by the processor and using a patient training model, one or more settings or thresholds that represent an expected level of coherence between the neural activity and the muscular activity or an expected level of neural activity and muscular activity to generate a movement of a portion of an avatar (block 440). The method may include determining, by the processor, the movement of the portion of an avatar based on the coherence or the relationship between the neural activity of the brain of the patient and the electrical activity of the muscle of the patient (block 450). The movement of the portion of the avatar may be further based on the one or more settings or thresholds. The movement of the portion of the avatar may be further based on the ratio of the neural activity and the ratio of the muscular activity at the different locations. The method may include rendering, by the processor and on a display, the movement of the portion of the avatar to provide biofeedback to the patient (block 460). The method may include communicating, by the processor, with a remote computing device to monitor the patient via the remote computing device, wherein monitoring the patient includes monitoring the detected neural activity of the brain of the patient or the detected muscular activity (block 470).

Exemplary embodiments of the methods, apparatus, and systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A neurofeedback rehabilitation system, comprising:
a plurality of sensors configured to detect at least one of neural activity of a brain of a patient and/or electrical activity of a muscle of the patient at different locations;
a display configured to render an image of a portion of an avatar that is representative of a patient; and
a processor coupled to the plurality of sensors and the display and configured to:
determine a movement of the portion of the avatar based on a coherence between the neural activity and the electrical activity of the patient at the different locations, and
render, on the display, the movement of the portion of the avatar to provide biofeedback to the patient.

2. The neurofeedback rehabilitation system of claim 1, further comprising:
a virtual reality, augmented reality, or mixed reality headset or other digital display configured to be placed over a head of the patient or in front of the patient and that includes the display.

3. The neurofeedback rehabilitation system of claim 1, wherein the processor is configured to:
determine at least one of a ratio of the neural activity at the different locations and a ratio of the muscular activity at the different locations; and
determine the movement of the portion of the avatar further based on the at least one of the ratio of the neural activity and the ratio of the muscular activity, as well as the neuromuscular coherence, at the different locations.

4. The neurofeedback rehabilitation system of claim 3, wherein the processor is configured to:
determine a change in the ratio of the neural activity at the different locations or a change in the ratio of the muscular activity at the different locations; and
determine a change in the movement of the portion of the avatar based on the change in the ratio of the neural activity or the change in the ratio of the muscular activity.

5. The neurofeedback rehabilitation system of claim 1, wherein the plurality of sensors include electroencephalography (EEG) sensors that are positioned at various locations on the head, wherein the EEG sensors are configured to detect the neural activity of the brain that corresponds to a movement or likely control of movement of a hand, an arm or a leg of the patient.

6. The neurofeedback rehabilitation system of claim 1, further comprising:
a sleeve including one or more sensors of the plurality of sensors, wherein the one or more sensors include one or more electromyography (EMG) sensors that are placed or fitted on the sleeve, wherein the one or more sensors are one or more flexible fabric electrodes or one or more disposable gel electrodes with snaps.

7. The neurofeedback rehabilitation system of claim 1, further comprising:
a communication interface configured to couple with a remote computing device that allows a provider to communicate with and monitor the patient, wherein monitoring the patient includes monitoring the detected neural activity of the brain of the patient or the detected muscular activity.

8. The neurofeedback rehabilitation system of claim 1, wherein the movement of the portion of the avatar is further based on one or more settings that are configurable by input received from a remote device that is operated by a provider, or by user input received via a user interface, or that is adjusted based on previously-detected neural activity or previously-detected muscular activity.

9. A portable, modular neurofeedback rehabilitation system, comprising:
a remote device configured to allow a provider to monitor or communicate with a patient;
a plurality of sensors configured to detect neural activity of a brain of a patient or electrical activity of a muscle of the patient at different locations;
a display configured to render an image of a portion of an avatar that is representative of a patient; and
a processor coupled to the plurality of sensors and the display and configured to:
determine a movement of the portion of the avatar based on a coherence of the neural activity of the brain and the electrical activity of the muscle of the patient, and
render, on the display, the movement of the portion of the avatar to provide biofeedback to the patient that results in an improvement of a condition of the patient or triggers a response by the patient.

10. The portable, modular neurofeedback rehabilitation system of claim 9, wherein the processor is configured to determine the movement of the portion of the avatar further based on one or more settings or thresholds that are personalized to the patient, wherein the remote device is configured to provide or adjust the one or more settings or thresholds that are personalized to the patient and the processor is configured to receive the one or more settings or thresholds, wherein the one or more settings or thresholds are adjusted over time to facilitate the improvement of the condition of the patient by requiring a stronger response or an increased amount of neural activity or electrical activity by the patient.

11. The portable, modular neurofeedback rehabilitation system of claim 9, further comprising one or more hand controllers configured to provide vibrotactile feedback to the patient in response to the virtual environment surrounding the avatar in the image and the detected neural activity and the detected muscular activity.

12. The portable, modular neurofeedback rehabilitation system of claim 9, wherein the plurality of sensors include at least one of an electroencephalography (EEG) sensor that is positioned at a location on a head of the patient, an electromyography sensor (EMG) sensor that is positioned at a location on a body part of the patient or a camera that tracks movement of an arm, a leg, a hand or other body part of the patient, wherein the EEG sensor(s) is configured to detect the neural activity of the brain that corresponds to a movement or likely control of movement of the arm, the leg, the hand or the other body part of the patient and the EMG sensor measures the electrical activity of the muscle of the body part of the patient.

13. The portable, modular neurofeedback rehabilitation system of claim 9, wherein the processor is configured to determine the movement of the portion of the avatar further based on an amplitude or magnitude of the neural activity or an amplitude or magnitude of the electrical activity generated by the muscle.

14. A method for providing biofeedback to a patient, comprising:
detecting, by a processor and using a plurality of sensors, neural activity of a brain of a patient and electrical activity of a muscle of the patient;
determining, by the processor, a movement of a portion of an avatar based on a coherence or relationship between the neural activity of the brain of the patient and the electrical activity of the muscle of the patient; and
rendering, by the processor and on a display, the movement of the portion of the avatar to provide biofeedback to the patient.

15. The method of claim 14, further comprising:
determining, by the processor, the coherence or the relationship between the neural activity of the brain of the patient and the electrical activity of the muscle of the patient.

16. The method of claim 14, further comprising:
determining, by the processor and using a patient training model, one or more settings or thresholds that represent an expected level of coherence between the neural activity and the muscular activity or an expected level of neural activity and muscular activity to generate the movement of the portion of the avatar.

17. The method of claim 16, wherein determining, by the processor, the movement of the portion of the avatar is further based on the one or more settings or thresholds.

18. The method of claim 14, further comprising:
obtaining, by the processor, patient data representative of a population of patients that require physical or occupational therapy after a condition including a cardiovascular stroke, wherein the patient data includes a level of coherence of neural activity of brains of the patients and electrical activity of muscles of the patients and corresponding settings or thresholds for movement of the portion of the avatar that induced improvement in movement of the patient; and
generating, by the processor, the patient training model using a machine learning algorithm based on the obtained patient data.

19. The method of claim 14, further comprising:
determining, by a processor, a ratio of the neural activity at the different locations and a ratio of the muscular activity at the different locations;
wherein determining the movement of the portion of the avatar is further based on the ratio of the neural activity and the ratio of the muscular activity at the different locations.

20. The method of claim 14, further comprising:
communicating, by the processor, with a remote computing device to monitor the patient via the remote computing device, wherein monitoring the patient includes monitoring the detected neural activity of the brain of the patient or the detected muscular activity.
